(19) United States
(12) Patent Application Publication
(10) Pub. No.: US 2023/0248416 A1
(43) Pub. Date: Aug. 10, 2023

(54) REUSABLE SURGICAL INSTRUMENTS HAVING USE COUNTING FUNCTIONALITY

(71) Applicant: Covidien LP, Mansfield, MA (US)

(72) Inventors: Arifmohamad Hamaju Mujawar, Sangli (IN); Naveen Kanth Nelli, Ambajipeta (IN); Rajanikanth Mandula, Hyderabad (IN); Priyanka Manepalli, Hyderabad (IN)

(21) Appl. No.: 17/665,023

(22) Filed: Feb. 4, 2022

(51) Int. Cl. A61B 18/14; A61B 18/12

(52) U.S. Cl. CPC A61B 18/1402; A61B 18/1206; A61B 2018/1412; A61B 2018/00601

(57) ABSTRACT

A surgical instrument includes a housing, an end effector extending distally from the housing, and a use counting feature disposed on the housing. The use counting feature includes a plurality of layers of material arranged in a stacked configuration such that only an upper-most layer of the plurality of layers of material is substantially visible. The upper-most layer of the plurality of layers of material is selectively removable from the plurality of layers of material such that, upon removal of the upper-most layer, a previously second upper-most layer of the plurality of layers of material becomes the upper-most layer and is substantially visible.

### FIELD

The present disclosure relates to surgical instruments and, more specifically, to reusable surgical instruments having use counting functionality.

### BACKGROUND

Surgical instruments may be generally classified as single-use instruments, reusable instruments, partially-reusable instruments (where one or more components are configured for single use and one or more other components are configured for reuse), or limited-use instruments (or instruments having limited-use components). Such limited-use instruments or components are classified as such because these instruments or components define useful lives during which use of the instruments or components is determined to be safe and effective. The useful life of a particular instrument or component is typically determined by testing and/or modeling that reflects the effects of the surgical environment on the instrument or component, stresses imparted to or by the instrument or component during use, the impact of sterilization and/or cleaning processes on the instrument or component, and/or the normal wear and tear resulting from repeated use of the instrument or component. The useful life of an instrument or component may be measured against one or more metrics such as: time from manufacturing, time in active use, time undergoing sterilization, number of activations, number of procedures, number of sterilizations, number of times assembled/disassembled, etc.

### SUMMARY

As used herein, the term “distal” refers to the portion that is being described which is farther from an operator, while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent. To the extent consistent, any of the aspects described herein may be used in conjunction with any or all of the other aspects described herein.

Provided in accordance with aspects of the present disclosure is a surgical instrument including a housing, an end effector extending distally from the housing, and a use counting feature disposed on the housing. The use counting feature includes a plurality of layers of material arranged in a stacked configuration such that only an upper-most layer of the plurality of layers of material is substantially visible. The upper-most layer of the plurality of layers of material is selectively removable from the plurality of layers of material such that, upon removal of the upper-most layer, a previously second upper-most layer of the plurality of layers of material becomes the upper-most layer and is substantially visible.

In an aspect of the present disclosure, the upper-most layer of the plurality of layers of material is manually removable, e.g., by peeling-off the upper-most layer.

In another aspect of the present disclosure, wherein the upper-most layer of the plurality of layers of material is automatically removable, e.g., in response to an event. In aspects, the event may be sterilization of the surgical instrument.

In still another aspect of the present disclosure, the upper-most layer of the plurality of layers of material is automatically removable by peeling-off the upper-most layer or by dissolving the upper-most layer.

In yet another aspect of the present disclosure, the plurality of layers of material are adhered to one another in the stacked configuration.

In still yet another aspect of the present disclosure, at least one layer of the plurality of layers of material is different in appearance from at least one other layer of the plurality of layers of material.

A surgical system provided in accordance with aspects of the present disclosure includes a surgical instrument and a test card. The surgical instrument includes a housing, an end effector extending distally from the housing, and a use counting feature disposed on the housing. The use counting feature includes a changing material configured to exhibit a change in an observable property thereof in response to at least one event associated with use of the surgical instrument. The test card includes a plurality of regions each including a different representation of the observable property to enable comparison of the observable property of the changing material with each of the representations to determine which representation matches or most closely matches the observable property of the changing material.

In an aspect of the present disclosure, the observable property of the changing material is color.

In another aspect of the present disclosure, the changing material is configured to exhibit a change in the observable property thereof in response to sterilization of the surgical instrument.

In still another aspect of the present disclosure, the changing material is configured to exhibit a change in the observable property thereof in response to at least one of: a temperature reaching a threshold temperature; a humidity reaching a threshold humidity; exposure to a fluid; or exposure to a cleaning chemical.

In yet another aspect of the present disclosure, the test card further includes indicia corresponding to the plurality of regions. The indicia may include an indicium corresponding to each region of the plurality of regions and indicating surgical instrument usage associated with that region.

Another surgical instrument provided in accordance with aspects of the present disclosure includes a housing defining a distal receptacle, an end effector insertable into the distal receptacle to releasably engage the end effector assembly with the housing, and a use counting feature disposed within the housing. The use counting feature includes a gear, a pawl, and a window. The gear extends partially into the distal receptacle, is rotatably mounted on a first transverse pin, and defines a plurality of one-way gear teeth disposed on an outer circumference thereof. Each gear tooth of the plurality of one-way gear teeth includes an indicium thereon. The pawl is initially engaged with a gear tooth of the plurality of one-way gear teeth to retain the gear in fixed rotational orientation. The pawl is rotatably mounted on a second transverse pin and biased into engagement with the gear tooth via a biasing spring. The window is defined though the housing to enable visualization of an indicum of the indicia that is aligned with the window from an exterior of the housing to provide an indication of surgical instrument usage. Upon insertion of the end effector into the distal receptacle, a portion of the end effector contacts the gear and rotates the gear such that the pawl rides over the engaged gear tooth to engage and adjacent gear tooth of the plurality of one-way gear teeth to thereby retain the gear in another fixed rotational orientation with a different indicum of the indicia visible through the window.

In an aspect of the present disclosure, each indicium indicates a number of uses or a number of uses remaining.

In another aspect of the present disclosure, the end effector is an electrosurgical blade. In such aspects, the surgical instrument may further include a plug assembly configured to connect the electrosurgical blade to an electrosurgical generator.

### BRIEF DESCRIPTION OF THE DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of an electrosurgical system including an electrosurgical generator and an electrosurgical pencil having a use counting feature in accordance with aspects of the present disclosure, wherein an end effector of the electrosurgical pencil is disengaged from a housing thereof;

FIGS. 2A and 2B are top views of the electrosurgical pencil of the system of FIG. 1 without and with the end effector, respectively;

FIG. 3A is an enlarged, perspective view of a distal portion of the housing of the electrosurgical pencil of the system of FIG. 1 including a use counting feature having selectively removable layers in accordance with aspects of the present disclosure;

FIG. 3B is an enlarged, perspective view of the distal portion of the housing of the electrosurgical pencil of the system of FIG. 1 including the use counting feature of FIG. 3A wherein the selectively removable layers are shown in an exploded condition;

FIG. 4 is an enlarged, perspective view of the distal portion of the housing of the electrosurgical pencil of the system of FIG. 1 including a use counting feature having a changing material with at least one changing observable property in accordance with aspects of the present disclosure;

FIG. 5 is a top view of a test card provided in accordance with aspects of the present disclosure including different representations of the observable property of the changing material of FIG. 4 to facilitate determination of use;

FIG. 6 is a longitudinal, cross-sectional view of a distal portion of the electrosurgical pencil of the system of FIG. 1 including another use counting feature in accordance with the present disclosure; and

FIGS. 7A and 7B are enlarged top and side internal, perspective views, respectively, of the distal portion of the electrosurgical pencil of the system of FIG. 1 illustrating the use counting feature of FIG. 6.

### DETAILED DESCRIPTION

Reusable surgical instruments provided in accordance with the present disclosure include use counting functionality to facilitate determination of the use status of a surgical instrument, e.g., to determine when a surgical instrument is approaching or has reached the end of its useful life. Although described hereinbelow with respect to an electrosurgical pencil, the aspects and features of the present disclosure are also contemplated for use with other suitable surgical instruments.

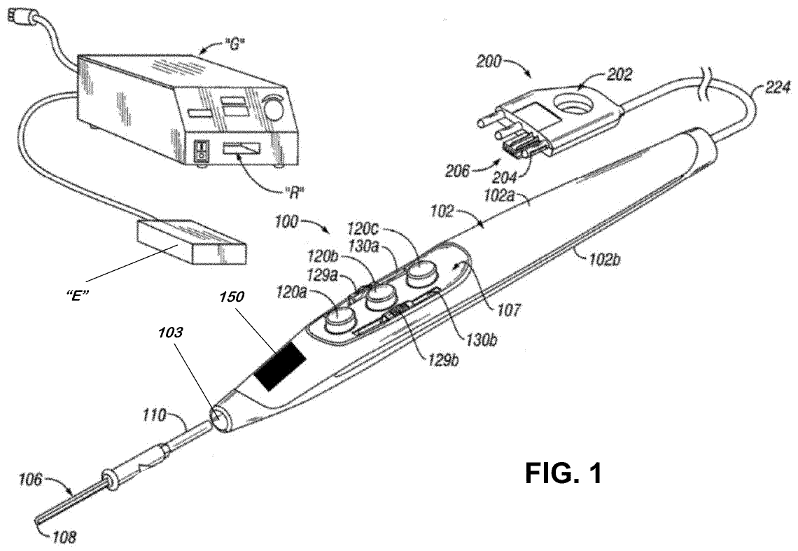

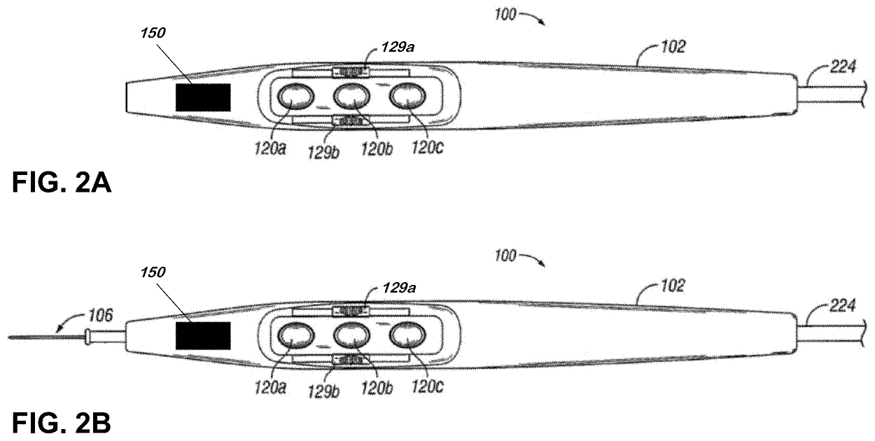

Turning to FIGS. 1-2B, an electrosurgical system provided in accordance with aspects of the present disclosure includes an electrosurgical pencil 100 and an electrosurgical generator “G.” Electrosurgical pencil 100 includes an elongated housing 102 formed from a first, e.g., top-half, housing portion 102a and a second, e.g., bottom-half, housing portion 102b. Housing 102 defines a distal receptacle 103 configured to releasably receive an end effector 106, and a proximal opening (not explicitly shown), through which a connecting cable 224 extends. With end effector 106 engaged within distal receptacle 103, a distal end portion 108 thereof protrudes distally from receptacle 103 while a proximal end portion 110 thereof is selectively retained by receptacle 103 within housing 102.

End effector 106 may be an electrosurgical blade, needle, loop, wire, wand, or any other suitable energy-based end effector configured to facilitate energy-based tissue treatment, e.g., cutting, cauterization, coagulation, etc. End effector 106 may be configured to supply monopolar Radio Frequency (RF) energy to tissue, may include isolated conductive portions to enable the supply of bipolar RF energy to tissue, or may supply another form of energy to tissue such as, for example, light energy, thermal energy, ultrasonic energy, microwave energy, etc.

Electrosurgical pencil 100 further includes a plug assembly 200 that electrically connects electrosurgical pencil 100 to electrosurgical generator “G.” Plug assembly 200 includes a housing 202 and connecting cable 224 interconnecting housing 202 of plug assembly 200 with housing 102 of the body of electrosurgical pencil 100. One or more pins 204 and/or contacts 206 of plug assembly 200 extends distally from housing 202 to enable electrical connection with electrosurgical generator “G” upon insertion of plug assembly 200 into receptacle “R” of electrosurgical generator “G.” Connecting cable 224 includes one or more energy supply wires that electrically couple to the one or more pins 204 and extend through connecting cable 224 into housing 102 to ultimately connect, directly or via suitable intermediate conductive elements, to end effector 106 when end effector 106 is engaged within distal receptacle 103 of housing 102. Connecting cable 224 further includes one or more control wires that electrically couple to the one or more contacts 206 and extend through connecting cable 224 into housing 102 to ultimately connect, directly or via suitable intermediate conductive elements, to various controls disposed on a user interface portion 107 of electrosurgical pencil 100 such as, for example, activation switches 120a-120c and/or intensity control sliders 129a, 129b movable along tracks 130a, 130b, respectively. In this manner, the user can selectively supply and control the application of electrosurgical energy from electrosurgical generator “G” to end effector 106 to achieve a desired tissue treatment therewith. In aspects where monopolar RF energy is delivered from end effector 106, a return pad electrode “E” or other suitable return device may be connected to electrosurgical generator “G” to enable the return of energy to complete the electrosurgical circuit.

Continuing with reference to FIGS. 1-2B, electrosurgical pencil 100 further includes a use counting feature 150, various configurations of which are detailed below. Use counting feature 150 may be configured, for example, for manually counting use (e.g., requiring user action to register a change in use) or for automatically counting use (e.g., registering a change in use based upon an event). The use (or remaining use) counting of use counting feature 150 may be temporal, event-based, and/or may count use continuously or discretely. Further, use counting feature 150 may provide an indication to the user as to use of electrosurgical pencil 100 (or a portion thereof) and/or available use (remaining use) of electrosurgical pencil 100 (or a portion thereof), e.g., relative to a useful life of electrosurgical pencil 100 (or the portion thereof). As an alternative or in addition to providing an indication, use counting feature 150 may communicate and/or interconnect with one or more other components of electrosurgical pencil 100 such as, for example, to break or disconnect components(s) of electrosurgical pencil 100, inhibit operable connection of electrosurgical pencil 100 to electrosurgical generator “G”, inhibit assembly of electrosurgical pencil 100, and/or otherwise render electrosurgical pencil 100 either partially or fully inoperable. Further still, although use counting feature 150 is shown disposed towards the distal end of housing 102 of electrosurgical pencil 100, it is contemplated that use counting feature 150 be disposed at any other suitable location such as, for example, at another location on or within housing 102, on housing 202 of plug assembly 200, etc.

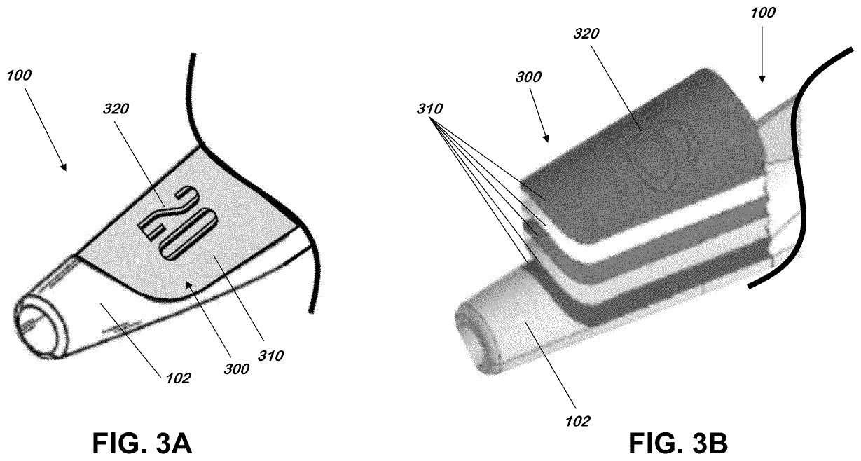

Turning to FIGS. 3A and 3B, in aspects, use counting feature 150 (FIGS. 1-2A) includes a plurality of layers of material 300 disposed on housing 102 of electrosurgical pencil 100. Each layer 310 of the plurality of layers of material 300 may be configured as a sheet with the lower-most layer 310 adhered or otherwise attached to housing 102 and each subsequent layer 310 stacked thereon and adhered or otherwise attached to the immediately below layer 310 in a stacked configuration. Each layer 310 may be visually different; for example, each layer 310 may define a different color, pattern, indicum, etc. With respect to indicia, each layer 310 may include a number indicator 320 indicating, for example, a number of uses or a number of remaining uses.

The upper-most layer 310 may be configured to be peeled-off from the immediately below layer 310 manually by a user to expose the immediately below layer 310. In this manner, the plurality of layers of material 300 may act as a stack of stickers to be removed sequentially from top to bottom by a user. The user may remove the upper-most layer 310 after each procedure, before or after each sterilization, upon each assembly, upon each disassembly, or in any other suitable manner to enable accurate use counting. Each layer 310, in aspects, may be formed from silicon, rubber, polytetrafluoroethylene (PTFE), or other suitable material. The layers 310 can be releasably bonded by a synthetic solvent-based adhesive or other suitable adhesive.

As an alternative to manual peeling, the upper-most layer 310 may automatically peel-off, dissolve, or otherwise be removed from the immediately below layer 310 in response to a certain event such as, for example, in response to heating of the upper-most layer 310 to a threshold temperature, upon reaching a threshold level of humidity in the surrounding environment, in response to interaction with particular fluid(s), cleaning chemical(s), etc. This is achieved, for example, by forming the upper-most layer 310 from a material and/or providing an adhesive material that is dissolvable at the threshold temperature, at the threshold humidity, by the particular fluid(s), cleaning chemical(s), etc. Further, while the layer material(s), adhesive material(s), etc. of the upper-most layer 310 are exposed to enable peeling-off or dissolving, the below layers 310, the adhesives thereof, etc., are nested, sealed-off, thermally insulated, and/or otherwise configured such that only the upper-most layer 310 is removed upon occurrence of each event while the below layers 310 remain stacked and attached with one another and electrosurgical pencil 100.

The event that results in peeling-off or dissolving the upper-most layer 310 may be, for example, sterilization or cleaning of electrosurgical pencil 100 and, thus, the upper-most layer 310 may automatically peel-off or dissolve from the immediately below layer 310 in response to sterilization and/or cleaning of electrosurgical pencil 100 such that use is counted by counting a number of sterilizations and/or cleanings. Sterilization and/or cleaning, for example, may involve high temperatures, high humidity, certain fluids, certain cleaning chemicals, certain materials, etc. that react with the material forming the upper-most layer 310 and/or the adhesive attaching the upper-most layer 310 to the below layer(s) 310 to enable the upper-most layer 310 peel-off or dissolve upon each sterilization and/or cleaning, thereby automatically counting each use.

As noted above, each layer 310 may include a number indicator 320 (or other suitable indicium) indicating, for example, a number of uses or a number of remaining uses. In such a configuration, only the number indicator 320 of the upper-most layer 310 is visible. However, upon peeling-off or dissolving of the upper-most layer 310, the previously immediately below layer 310 becomes the new upper-most layer 310 with only its number indicator 320 now visible. Thus, use counting is readily achieved. As an alternative to number indicators 320, other suitable indicators can be provided such as symbols, icons, colors, etc. In aspects, the various layers 310 need not all have different indicators but, rather, can be grouped into several groups, for example: an upper-most group of one or more layers 310 each providing a green indicator and/or a check-mark indicator; a middle group of one or more layers 310 each providing a yellow indicator and/or caution sign indicator; and a lower group of one or more layers 310 each providing a red indicator and/or stop sign indicator. In aspects, the lower group of layers 310 may include just one layer 310; additional or alternative layers or groups thereof with various different indicators to indicate an amount of use or an amount of remaining use of electrosurgical pencil 100 at any suitable level of granularity are also contemplated.

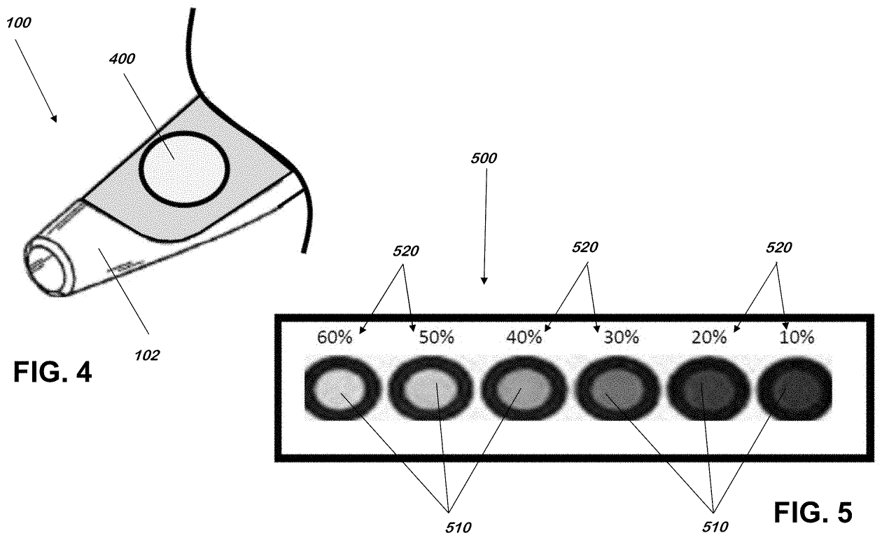

Referring to FIG. 4, in aspects, use counting feature 150 (FIGS. 1-2A) includes a changing material 400 disposed on, incorporated as part of, and/or disposed within housing 102 of electrosurgical pencil 100. Changing material 400 is configured to change at least one observable property thereof such as, for example: a color or shade of the material, a pattern of the material; a texture of the material; a thickness of the material; etc. The observable property of changing material 400 may be configured to change in response to one or more events and may change continuously or discretely; uniformly or in any other suitable manner. For example, the observable property of changing material 400 may change throughout successive events in a progressive manner such as wherein a color is successively darkened, wherein a thickness is successively decreased, wherein a pattern is successively enlarged, wherein a texture is successively amplified, etc.

The event resulting in change to the observable property of the changing material 400 may include, for example, heating of the changing material 400 to a threshold temperature, upon reaching a threshold level of humidity in the surrounding environment, in response to interaction with particular fluid(s), cleaning chemical(s), etc. Further, the event that results in change to the observable property of changing material 400 may be, for example, sterilization or cleaning of electrosurgical pencil 100 (similarly as detailed above) and, thus, the change to the observable property of changing material 400 may be utilized to indicate when sterilization and/or cleaning of electrosurgical pencil 100 has occurred such that use is counted by counting a number of sterilizations and/or cleanings.

With additional reference to FIG. 5, in aspects, a test card 500 may be provided to facilitate evaluation of the use counting provided by the observable property of the changing material 400. For example, test card 500 may include a plurality of different regions 510 each providing a representation of the observable property as it would be observed at a particular amount of use or amount of use remaining. Test card 500 may further include an indicator 520 corresponding to each region 510, wherein the indicator 520 indicates the particular amount of use or amount of use remaining for the corresponding representation of the observable property. For example, regions 510 may provide representations of the color of the changing material 400 at different percentages of use remaining and each indicator 520 may indicate the corresponding percentage of use remaining for each region 510. As such, a user can compare the present color (or other observable property) of the changing material 400 on electrosurgical pencil 100 with the matching (or most closely matching) representative region 510 on test card 500 to readily determine the use count, e.g., the percentage of remaining use, of electrosurgical pencil 100.

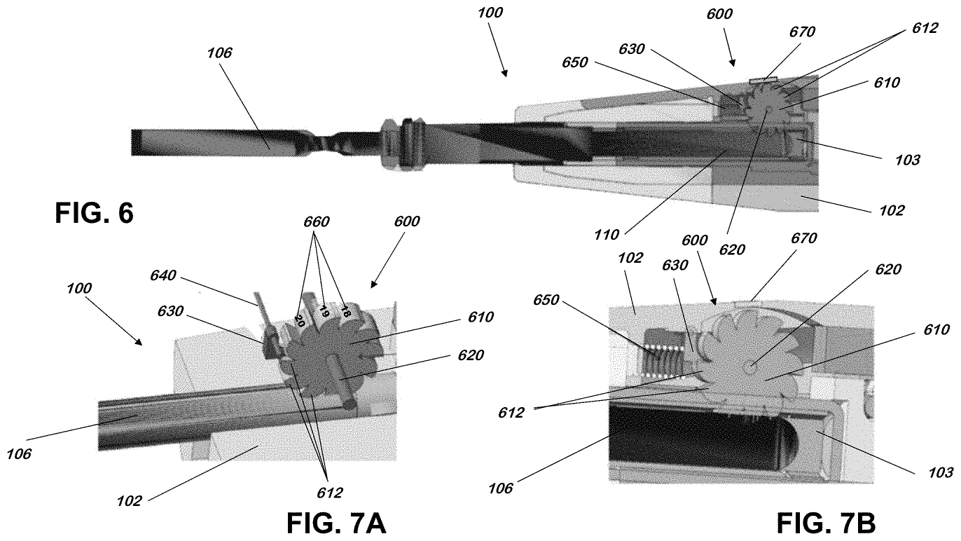

Referring to FIGS. 6 and 7A-7B, in aspects, use counting feature 150 (FIGS. 1-2A) includes a counting mechanism 600 incorporated into electrosurgical pencil 100 and configured to increment each time an end effector 106 is engaged within distal receptacle 103 of housing 102. Counting mechanism 600 includes a gear 610 having a plurality of one-way gear teeth 612 disposed annularly about a circumference thereof; a transverse pin 620 rotatably supporting gear 610 within housing 102 of electrosurgical pencil 100 such that gear 610 at least partially protrudes into distal receptacle 103; a pawl 630 configured to incrementally engage one-way gear teeth 612 as gear 610 is rotated relative to pawl 630; a transverse pin 640 rotatably supporting pawl 630; and a biasing spring 650 biasing pawl 630 into operable engagement with gear 610. Counting mechanism 600 further includes indicia 660 (e.g., number indicators indicating a number of uses or a number of uses remaining) disposed on one-way gear teeth 612, and a window 670 disposed in housing 102 to permit visualization of the indicium 660 of the one-way gear tooth 612 aligned with window 670 from the exterior of housing 102.

Initially, pawl 630, under the bias of spring 650, retains gear 610 in fixed rotational orientation relative to housing 102. Upon insertion of end effector 106 into distal receptacle 103 of housing 102 to engage proximal end portion 110 of end effector 106 therein (and to electrically connect end effector 106 to the corresponding electrical connectors within housing 102), proximal end portion 110 of end effector 106 contacts a portion of gear 610 that protrudes into distal receptacle 103 of housing 102, thereby urging gear 610 to rotate one increment about pin 620 to a next rotational orientation. As gear 610 is rotated in this manner, pawl 630 rides over the previously-engaged one-way gear tooth 612 until pawl 630 engages an adjacent one-way gear tooth 612 to retain gear 610 in the next rotational orientation relative to housing 102. Pawl 630, in conjunction with the one-way configuration of gear teeth 612, inhibit reverse rotation of gear 610, e.g., in response to disengagement and removal of end effector 106 from distal receptacle 103 of housing 102. However, upon each re-insertion of end effector 106 or insertion of another end effector 106, the proximal end portion 110 of end effector 106 again contacts a portion of gear 610 that protrudes into distal receptacle 103 of housing 102, thereby urging gear 610 to rotate another increment about pin 620 to a further rotational orientation.

At each rotational orientation of gear 610, a different indicum 660 is aligned with and visible through window 670. In this manner, by viewing the indicium 660 visible through window 670, the use of electrosurgical pencil 100, e.g., the number of uses or number of uses remaining, can be readily determined. In aspects, the indicum 660 may all be different, e.g., as numbers, or may be grouped into plural groups with each group having one or more gear teeth 612 with the same indicium 660 (similarly as detailed above).

While several aspects of the present disclosure have been shown in the drawings, it is not intended that the present disclosure be limited thereto, as it is intended that the present disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

What is claimed is:

1. A surgical instrument, comprising:
a housing;
an end effector extending distally from the housing; and
a use counting feature disposed on the housing, the use counting feature including a plurality of layers of material arranged in a stacked configuration such that only an upper-most layer of the plurality of layers of material is substantially visible, the upper-most layer of the plurality of layers of material selectively removable from the plurality of layers of material such that, upon removal of the upper-most layer, a previously second upper-most layer of the plurality of layers of material becomes the upper-most layer and is substantially visible.

2. The surgical instrument according to claim 1, wherein the upper-most layer of the plurality of layers of material is manually removable.

3. The surgical instrument according to claim 2, wherein the upper-most layer of the plurality of layers of material is manually removable by peeling-off the upper-most layer.

4. The surgical instrument according to claim 1, wherein the upper-most layer of the plurality of layers of material is automatically removable in response to an event.

5. The surgical instrument according to claim 4, wherein the event is sterilization of the surgical instrument.

6. The surgical instrument according to claim 4, wherein the upper-most layer of the plurality of layers of material is automatically removable by peeling-off the upper-most layer.

7. The surgical instrument according to claim 4, wherein the upper-most layer of the plurality of layers of material is automatically removable by dissolving the upper-most layer.

8. The surgical instrument according to claim 1, wherein the plurality of layers of material are adhered to one another in the stacked configuration.

9. The surgical instrument according to claim 1, wherein at least one layer of the plurality of layers of material is different in appearance from at least one other layer of the plurality of layers of material.

10. A surgical system, comprising:
a surgical instrument, including:
a housing;
an end effector extending distally from the housing; and
a use counting feature disposed on the housing, the use counting feature including a changing material, wherein the changing material is configured to exhibit a change in an observable property thereof in response to at least one event associated with use of the surgical instrument; and
a test card including a plurality of regions, each region of the plurality of regions including a different representation of the observable property to enable comparison of the observable property of the changing material with each of the representations to determine which representation matches or most closely matches the observable property of the changing material.

11. The surgical system according to claim 10, wherein the observable property of the changing material is color.

12. The surgical system according to claim 10, wherein the changing material is configured to exhibit a change in the observable property thereof in response to sterilization of the surgical instrument.

13. The surgical system according to claim 10, wherein the changing material is configured to exhibit a change in the observable property thereof in response to at least one of: a temperature reaching a threshold temperature; a humidity reaching a threshold humidity; exposure to a fluid; or exposure to a cleaning chemical.

14. The surgical system according to claim 10, wherein the test card further includes indicia corresponding to the plurality of regions.

15. The surgical system according to claim 14, wherein the indicia includes an indicium corresponding to each region of the plurality of regions and indicating surgical instrument usage associated with that region.

16. A surgical instrument, comprising:
a housing defining a distal receptacle;
an end effector insertable into the distal receptacle to releasably engage the end effector assembly with the housing; and
a use counting feature disposed within the housing, the use counting feature including:
a gear extending partially into the distal receptacle, the gear rotatably mounted on a first transverse pin and defining a plurality of one-way gear teeth disposed on an outer circumference thereof, each gear tooth of the plurality of one-way gear teeth including an indicium thereon;
a pawl initially engaging a gear tooth of the plurality of one-way gear teeth to retain the gear in fixed rotational orientation, the pawl rotatably mounted on a second transverse pin and biased into engagement with the gear tooth via a biasing spring; and
a window defined though the housing to enable visualization of an indicum of the indicia that is aligned with the window from an exterior of the housing to provide an indication of surgical instrument usage,
wherein, upon insertion of the end effector into the distal receptacle, a portion of the end effector contacts the gear and rotates the gear such that the pawl rides over the engaged gear tooth to engage and adjacent gear tooth of the plurality of one-way gear teeth to thereby retain the gear in another fixed rotational orientation with a different indicum of the indicia visible through the window.

17. The surgical instrument according to claim 16, wherein each indicium indicates a number of uses or a number of uses remaining.

18. The surgical instrument according to claim 16, wherein the end effector is an electrosurgical blade.

19. The surgical instrument according to claim 18, further comprising a plug assembly configured to connect the electrosurgical blade to an electrosurgical generator.
